(19) United States
(12) Patent Application Publication
(10) Pub. No.: US 2025/0331809 A1
(43) Pub. Date: Oct. 30, 2025

(54) MEDICAL PROCESSING APPARATUS AND MEDICAL PROCESSING METHOD

(71) Applicants: CANON MEDICAL SYSTEMS CORPORATION, Otawara-shi (JP); SHOWA UNIVERSITY, Tokyo (JP)

(72) Inventors: Seina AMAGI, Nasushiobara (JP); Kiyotake ICHIZUKA, Shinagawa-ku (JP)

(73) Assignees: CANON MEDICAL SYSTEMS CORPORATION, Otawara-shi (JP); SHOWA UNIVERSITY, Tokyo (JP)

(21) Appl. No.: 19/187,215

(22) Filed: Apr. 23, 2025

(30) Foreign Application Priority Data
Apr. 24, 2024 (JP) 2024-070784

(51) Int. Cl. A61B 8/08; A61B 8/00

(52) U.S. Cl. CPC A61B 8/0866; A61B 8/466; A61B 8/483

(57) ABSTRACT

A medical processing apparatus according to the present embodiment comprises a processing circuitry configured to acquire three-dimensional volume data of a subject, extract a first section intersecting a reference line of the subject included in the three-dimensional volume data, extract a second section intersecting the first section from the three-dimensional volume data based on a posture of the subject included in the first section, and output information based on the second section.

### CROSS REFERENCE TO RELATED APPLICATIONS

This application is based upon and claims the benefit of priority from the prior Japanese Patent Application No. 2024-070784, filed on Apr. 24, 2024, the entire contents of which are incorporated herein by reference.

### FIELD

Embodiments described in the present specification and drawings relate to a medical processing apparatus and a medical processing method.

### BACKGROUND

In the examination of a fetus, it is necessary to extract a median sagittal section, which is a plane dividing a subject into left and right symmetry (both halves), from three-dimensional volume data in order to measure a crown-rump length (CRL) of the fetus, a nuchal translucency (NT) region of the fetus, a nasal bone (NB) of the fetus, and the like. In addition, in addition to the extraction of the median sagittal section in the examination of the fetus, a section (hereinafter, referred to as a standard section) necessary for measurement may be extracted from three-dimensional volume data in a case where measurement of a long axis length, a long axis sectional area, and the like of a corpus callosum or a left ventricle at an end stage of diastole of the subject is performed on a two-dimensional image.

In related art, the standard section such as the median sagittal section is extracted from the three-dimensional volume data by a user searching for the standard section from the three-dimensional volume data. In addition, in recent years, in order to reduce time and effort of the user searching for the standard section from the three-dimensional volume data, the standard section is automatically extracted from the three-dimensional volume data by using a technique of fully searching for the standard section from all azimuthal sections in the three-dimensional volume data or a technique of searching for the standard section using artificial intelligence (AI).

However, in a case where the standard section is extracted by using the technique of fully searching for the standard section from all the azimuthal sections in three-dimensional volume data, there is a possibility that enormous time is required for the processing. In addition, in a case where the standard section is extracted by using the technique of searching for the standard section using the artificial intelligence (AI), it is difficult to extract the standard section from the three-dimensional volume data in a case where there is little difference in features between a section to be extracted in the three-dimensional volume data and a neighboring section thereof. Thus, it is desired that the user can easily extract the standard section of the subject from the three-dimensional volume data.

### BRIEF DESCRIPTION OF THE DRAWINGS

FIG. 1 is a block diagram illustrating an example of a configuration of a medical processing system according to a first embodiment;

FIG. 2 is a flowchart for explaining information output processing executed in a medical processing apparatus according to the first embodiment;

FIG. 3 is a diagram illustrating an example of three-dimensional volume data according to the first embodiment;

FIG. 4 is a diagram illustrating an example of a measurement target of a subject according to the first embodiment;

FIG. 5 is a diagram for explaining processing of extracting a first section from a plurality of sections;

FIG. 6A is a diagram illustrating one section intersecting a reference line. FIG. 6B is a diagram illustrating another section intersecting the reference line;

FIG. 7A is a diagram illustrating a region representing a fetus included in the section illustrated in FIG. 6A. FIG. 7B is a diagram illustrating a region representing the fetus included in the section illustrated in FIG. 6B;

FIG. 8 is a diagram for explaining processing of detecting a posture of the fetus;

FIG. 9 is a diagram for explaining processing of extracting a second section;

FIG. 10 is a diagram illustrating an example of information based on the second section;

FIG. 11 is a flowchart for explaining information output processing according to Modification 1;

FIG. 12A is a diagram illustrating a third ventricle. FIG. 12B is a diagram illustrating a sacrococcygeal part;

FIG. 13A is a diagram illustrating feature points of the third ventricle illustrated in FIG. 12A. FIG. 13B is a diagram illustrating feature points of the sacrococcygeal part illustrated in FIG. 12B;

FIG. 14 is a flowchart for explaining information output processing executed in a medical processing apparatus according to a second embodiment;

FIG. 15 is a diagram illustrating an example of three-dimensional volume data according to the second embodiment;

FIG. 16 is a diagram illustrating an example of a measurement target of a subject according to the second embodiment;

FIG. 17 is a diagram illustrating an example of a section including orbits;

FIG. 18 is a diagram for explaining processing of detecting a posture of a head;

FIG. 19 is a diagram for explaining processing of extracting a second section;

FIG. 20 is a block diagram illustrating an example of a configuration of a medical processing system according to Modification 2;

FIG. 21 is a flowchart for explaining information output processing executed in a medical processing apparatus according to Modification 2; and

FIG. 22 is a diagram illustrating an example of a bilaterally symmetrical anatomical structure according to Modification 2.

### DETAILED DESCRIPTION

Hereinafter, respective embodiments of the medical processing apparatus and the medical processing method will be described with reference to the accompanying drawings. In the embodiments below, the same reference signs are given for identical components in terms of configuration and function, and duplicate description is omitted.

### First Embodiment

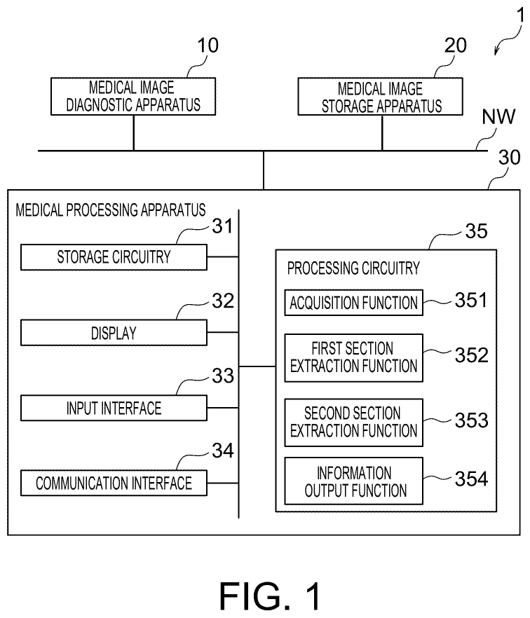

FIG. 1 is a block diagram illustrating an example of a configuration of a medical processing system according to a first embodiment. As illustrated in FIG. 1, a medical processing system 1 includes a medical image diagnostic apparatus 10, a medical image storage apparatus 20, and a medical processing apparatus 30. The medical image diagnostic apparatus 10, the medical image storage apparatus 20, and the medical processing apparatus 30 are connected to communicate with each other via an in-hospital network NW by a dedicated line in a hospital.

Note that, the medical image diagnostic apparatus 10, the medical image storage apparatus 20, and the medical processing apparatus 30 may be connected to communicate with each other via a network via a public line such as the Internet. In addition, although the medical processing apparatus 30 is provided separately from the medical image diagnostic apparatus 10, the medical processing apparatus 30 may be provided integrally with the medical image diagnostic apparatus 10.

The medical image diagnostic apparatus 10 images a subject and generates medical data. The medical image diagnostic apparatus 10 transmits the generated medical data to the medical image storage apparatus 20 or the medical processing apparatus 30 via the in-hospital network NW. For example, the medical image diagnostic apparatus 10 is an ultrasonic diagnostic apparatus, an X-ray computed tomography (CT) apparatus, a magnetic resonance imaging (MRI) apparatus, an X-ray diagnostic apparatus, a PET apparatus, a SPECT apparatus, or the like. In addition, for example, the medical data generated by the medical image diagnostic apparatus 10 is three-dimensional volume data. In the following description, a case where the medical image diagnostic apparatus 10 is the ultrasonic diagnostic apparatus will be described as an example.

The medical image storage apparatus 20 stores the medical data generated by the medical image diagnostic apparatus 10, various types of data generated by the medical processing apparatus 30, and the like. In addition, the medical image storage apparatus 20 transmits the stored medical data, various types of data, and the like to the medical image diagnostic apparatus 10 or to the medical processing apparatus 30 via the in-hospital network NW. For example, the medical image storage apparatus 20 is an image server such as a picture archiving and communication system (PACS). In addition, the medical image storage apparatus 20 may be realized by a server group (cloud) connected to the medical processing system 1 via a network.

The medical processing apparatus 30 performs various types of information processing regarding the subject. Specifically, the medical processing apparatus 30 executes various types of processing by using three-dimensional volume data that is medical data generated by the ultrasonic diagnostic apparatus that is the medical image diagnostic apparatus 10 and three-dimensional volume data transmitted from the medical image storage apparatus 20. For example, the medical processing apparatus 30 is realized by a computer device such as a server or a workstation.

As illustrated in FIG. 1, the medical processing apparatus 30 includes a storage circuitry 31, a display 32, an input interface 33, a communication interface 34, and a processing circuitry 35.

The storage circuitry 31 is realized by, for example, a random access memory (RAM), a semiconductor memory element such as a flash memory, a hard disk, an optical disk, or the like. The storage circuitry 31 stores, for example, the three-dimensional volume data generated by the medical image diagnostic apparatus 10, the three-dimensional volume data transmitted from the medical image storage apparatus 20, various types of data generated by the medical processing apparatus 30, and the like.

The display 32 displays various types of data and information. For example, the display 32 displays a graphical user interface (GUI) or the like for receiving various operations from a user. The display 32 includes, for example, a liquid crystal display, a cathode ray tube (CRT) display, or the like.

The input interface 33 receives various input operations from the user, converts the received input operations into electric signals, and outputs the electric signals to the processing circuitry 35. The input interface 33 is realized by, for example, a mouse, a keyboard, a trackball, a manual switch, a foot switch, a button, a joystick, a touch pad that performs an input operation by touching an operation surface, a touch screen in which a display screen and the touch pad are integrated, a non-contact input interface using an optical sensor, a voice input interface, and the like. Note that, in the present specification, the input interface 33 is not limited to the interface including physical operation components such as a mouse and a keyboard. For example, an electric signal processing circuitry that receives an electric signal corresponding to an input operation from an external input device provided separately from the apparatus and outputs the electric signal to a control circuitry is included in examples of the input interface 33.

The communication interface 34 implements various types of information communication protocols corresponding to the form of the in-hospital network NW. The communication interface 34 realizes communication with other devices via the in-hospital network NW according to various protocols. The medical processing apparatus 30 is connected to the in-hospital network NW via the communication interface 34, and communication with the medical image diagnostic apparatus 10 and the medical image storage apparatus 20 is realized.

In addition, the processing circuitry 35 is an arithmetic circuitry that performs various arithmetic operations, and includes, for example, a processor such as a CPU or a GPU. The processing circuitry 35 acquires the three-dimensional volume data from the medical image diagnostic apparatus 10 or the medical image storage apparatus 20, extracts a first section from the three-dimensional volume data, extracts a second section from the three-dimensional volume data, or outputs information based on the second section, for example. The information based on the second section is, for example, an image based on the second section extracted from the three-dimensional volume data.

Thus, the processing circuitry 35 has an acquisition function 351, a first section extraction function 352, a second section extraction function 353, and an information output function 354. The acquisition function 351 corresponds to an acquisition unit according to the present embodiment, the first section extraction function 352 corresponds to a first section extraction unit according to the present embodiment, the second section extraction function 353 corresponds to a second section extraction unit according to the present embodiment, and the information output function 354 corresponds to an information output unit according to the present embodiment.

Each processing function performed by the acquisition function 351, the first section extraction function 352, the second section extraction function 353, and the information output function 354 is stored in the storage circuitry 31 in the form of a program executable by the computer. The processing circuitry 35 is a processor that realizes a function corresponding to each program by reading and executing the program from the storage circuitry 31. In other words, the processing circuitry 35 that has read each program has each function illustrated in the processing circuitry 35 of FIG. 1. Note that, in FIG. 1, although it has been described that the acquisition function 351, the first section extraction function 352, the second section extraction function 353, and the information output function 354 are realized by the single processing circuitry 35, these functions may be realized by combining a plurality of independent processors to constitute the processing circuitry 35 and executing a program by each processor.

The acquisition function 351 acquires the three-dimensional volume data of the subject. The first section extraction function 352 extracts a first section intersecting a reference line of the subject included in the three-dimensional volume data.

The second section extraction function 353 extracts a second section intersecting the first section from the three-dimensional volume data based on a posture of the subject included in the first section. The information output function 354 outputs information based on the second section.

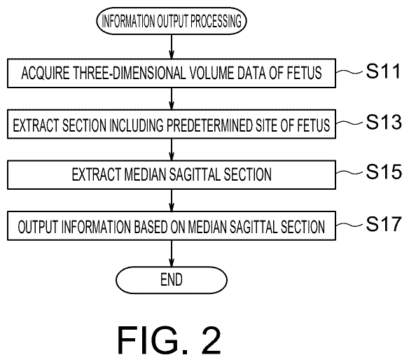

FIG. 2 is a flowchart for explaining information output processing executed in the medical processing apparatus 30 according to the first embodiment. In this information output processing, three-dimensional volume data of a fetus that is the subject is acquired from the medical image diagnostic apparatus 10 or the medical image storage apparatus 20, a section including a predetermined site of the fetus is extracted as the first section, a median sagittal section is extracted as the second section, or information based on the median sagittal section is output. For example, the information output processing is processing executed in a case where the three-dimensional volume data is acquired from the medical image diagnostic apparatus 10 or the medical image storage apparatus 20.

As illustrated in FIG. 2, first, the acquisition function 351 in the processing circuitry 35 of the medical processing apparatus 30 acquires the three-dimensional volume data of the fetus (step S11). Specifically, the acquisition function 351 acquires the three-dimensional volume data of the fetus from the medical image diagnostic apparatus 10 or the medical image storage apparatus 20 via the communication interface 34. The acquisition function 351 may directly acquire the three-dimensional volume data of the fetus via the communication interface 34, or the acquisition function 351 in the processing circuitry 35 may acquire the three-dimensional volume data of the fetus from the storage circuitry 31 after temporarily storing the three-dimensional volume data acquired via the communication interface 34 in the storage circuitry 31.

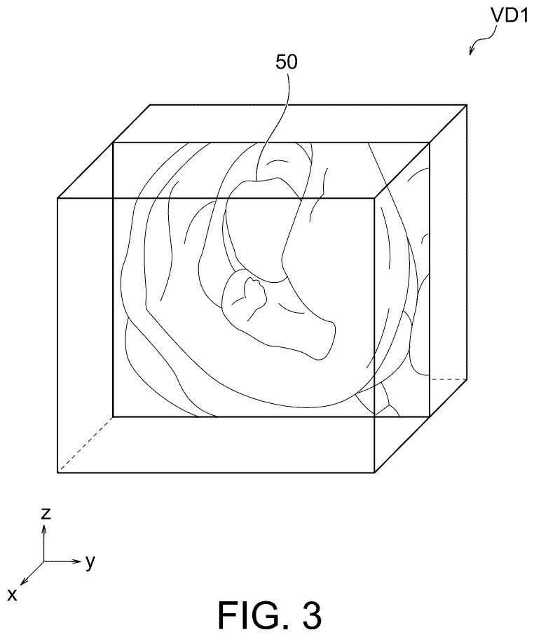

FIG. 3 is a diagram illustrating an example of the three-dimensional volume data according to the first embodiment. As illustrated in FIG. 3, the acquisition function 351 acquires three-dimensional volume data VD1 of the fetus. In the example illustrated in FIG. 3, the three-dimensional volume data VD1 of the fetus includes a whole body 50 of the fetus and the like.

Subsequently, as illustrated in FIG. 2, the first section extraction function 352 in the processing circuitry 35 of the medical processing apparatus 30 extracts a section including a predetermined site of the fetus (step S13). Specifically, the first section extraction function 352 extracts a first section intersecting a reference line and including a predetermined site of the fetus. More specifically, the first section extraction function 352 acquires the reference line based on a measurement target of the fetus that is a subject included in the three-dimensional volume data VD1, and extracts, as the first section, a section intersecting the reference line and including a predetermined site of the fetus.

Hereinafter, a method for extracting the first section in the medical processing apparatus 30 will be described.

In step S13, first, the first section extraction function 352 detects the measurement target of the fetus included in the three-dimensional volume data VD1 acquired in step S11. Specifically, the first section extraction function 352 detects, as the measurement target, a crown-rump length of the fetus included in the three-dimensional volume data VD1. More specifically, the first section extraction function 352 detects the crown-rump length of the fetus by inputting the three-dimensional volume data VD1 to a trained model, detecting a region from the head to the torso in the three-dimensional volume data VD1 by the trained model, and obtaining end points of the crown-rump length from the region by the logic.

That is, the trained model is a model that has learned to detect a predetermined region in the three-dimensional volume data VD1 in response to the input of the three-dimensional volume data VD1. In the present embodiment, this trained model is a model that has learned to detect, as the predetermined region, the region from the head to the torso in the three-dimensional volume data VD1. In addition, the logic is a logic for obtaining information regarding the measurement target of the subject from the predetermined region. In the present embodiment, this logic is a logic for obtaining the end points of the crown-rump length from the region from the head to the torso, which is the predetermined region, as the information regarding the measurement target of the subject.

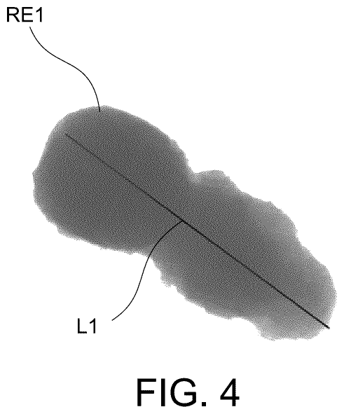

FIG. 4 is a diagram illustrating an example of the measurement target of the subject according to the first embodiment. As illustrated in FIG. 4, the first section extraction function 352 detects a region RE1 of the torso from the head of the fetus from the three-dimensional volume data VD1 by using the trained model, obtains, as the end points of the crown-rump length, two positions positioned at a boundary of the detected region RE1 of the torso from the head of the fetus and having a maximum distance from each other by using the logic, and detects a line segment connecting the two positions as a crown-rump length L1 of the fetus.

Subsequently, in step S13, the first section extraction function 352 acquires the reference line based on the measurement target of the fetus included in the three-dimensional volume data VD1. Specifically, the first section extraction function 352 acquires the reference line based on the crown-rump length L1, which is the measurement target detected from the three-dimensional volume data VD1. More specifically, the first section extraction function 352 acquires the reference line by setting the crown-rump length L1 in the three-dimensional volume data VD1 based on the crown-rump length L1 that is the measurement target detected from the three-dimensional volume data VD1.

Subsequently, in step S13, the first section extraction function 352 extracts the first section intersecting the reference line. Specifically, the first section extraction function 352 extracts, as the first section, the section including the predetermined site of the fetus, among a plurality of sections intersecting a plurality of different positions of the reference line acquired based on the crown-rump length L1. More specifically, the first section extraction function 352 extracts, as the first section, a section including the head, which is the predetermined site of the fetus, among the plurality of sections intersecting the plurality of different positions of the reference line acquired based on the crown-rump length L1. In order to extract the section including the head of the fetus, the first section extraction function 352 extracts, as the first section, a section having a largest region representing the fetus, among the plurality of sections intersecting the plurality of different positions of the reference line. In addition, in a case where the plurality of sections intersecting the plurality of different positions of the reference line are extracted from the three-dimensional volume data VD1, the first section extraction function 352 extracts the plurality of sections intersecting the plurality of different positions of the reference line such that directions intersecting the reference line are equal, that is, the sections are parallel to each other.

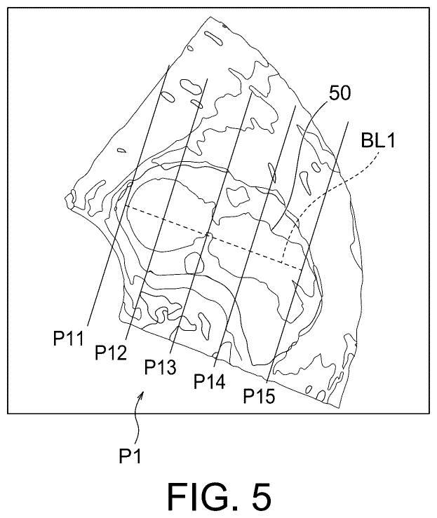

FIG. 5 is a diagram for explaining processing of extracting the first section from the plurality of sections. As illustrated in FIG. 5, the first section extraction function 352 equally divides a reference line BL1 into a plurality of parts, and extracts sections orthogonal to the reference line BL1 at positions where the head of the fetus is estimated to be positioned in the whole body 50 of the fetus from the three-dimensional volume data VD1, among a plurality of positions P1 obtained by equally dividing the reference line BL1 into the plurality of parts. In the example illustrated in FIG. 5, the first section extraction function 352 equally divides the reference line BL1 into four parts, and extracts, from the three-dimensional volume data VD1, sections orthogonal to the reference line BL1 at two different positions P12 and P14 at which the head of the fetus is estimated to be positioned, among five positions P1 obtained by equally dividing the reference line BL1 into four parts. That is, in the example illustrated in FIG. 5, the first section extraction function 352 extracts two sections orthogonal to two different positions P12 and P14 of the reference line BL1 from the three-dimensional volume data VD1. The two sections orthogonal to the two different positions of the reference line BL1 have the same direction orthogonal to the reference line BL1, that is, are parallel to each other.

Note that, although it has been described that the first section extraction function 352 extracts, from the three-dimensional volume data VD1, the sections orthogonal to the reference line BL1 at the two different positions P12 and P14 at which the head of the fetus is estimated to be positioned, among the positions P1 obtained by equally dividing the reference line BL1 into four parts, the first section extraction function 352 may extract, from the three-dimensional volume data VD1, the sections orthogonal to the reference line BL1 at the plurality of positions P1 obtained by equally dividing the reference line BL1 into a plurality of parts.

In addition, each of the plurality of sections intersecting the plurality of different positions of the reference line BL1 is not limited to a case where the directions intersecting the reference line BL1 are equal. That is, the plurality of sections intersecting the plurality of different positions of the reference line BL1 may be substantially equal in direction intersecting the reference line BL1, that is, substantially parallel to each other.

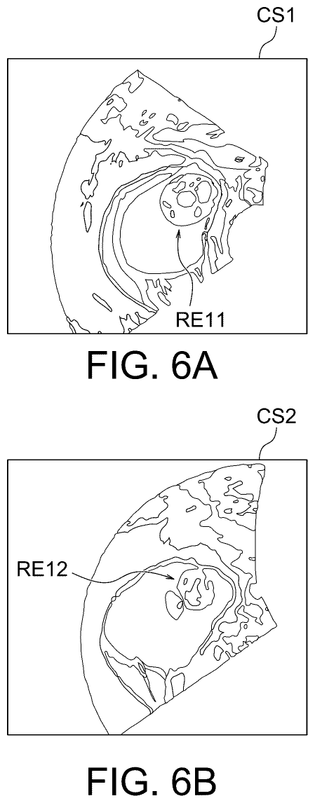

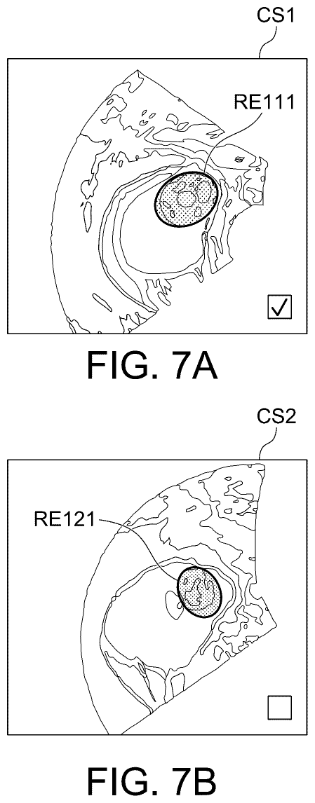

FIGS. 6A and 6B is a diagram illustrating the section intersecting the reference line. FIGS. 7A and 7B is a diagram illustrating the region representing the fetus included in the section. In the example illustrated in FIG. 6A, a region RE11 representing the fetus is drawn in a section CS1 at the position P12 illustrated in FIG. 5. In addition, in the example illustrated in FIG. 6B, a region RE12 representing the fetus is drawn in a section CS2 at the position P14 illustrated in FIG. 5. As illustrated in FIGS. 7A and 7B, the first section extraction function 352 elliptically fits the regions RE11 and RE12 representing the fetus in the plurality of sections CS1 and CS2 extracted from the three-dimensional volume data VD1 by using an elliptical template. As illustrated in FIGS. 7A and 7B, the first section extraction function 352 extracts, as the first section, the section CS1 having a largest region representing the fetus, among elliptically fitted regions RE111 and RE121 representing the fetus in the section CS1 and the section CS2. In this manner, the first section extraction function 352 extracts, as the first section, the section CS1 intersecting the reference line BL1 and including the head of the fetus.

Subsequently, as illustrated in FIG. 2, the second section extraction function 353 in the processing circuitry 35 of the medical processing apparatus 30 extracts a median sagittal section (step S15). Specifically, the second section extraction function 353 extracts the median sagittal section intersecting the section CS1 including the head of the fetus from the three-dimensional volume data VD1 based on the posture of the fetus included in the section including the head of the fetus. More specifically, the second section extraction function 353 detects an orientation of the head of the fetus as the posture of the fetus, and extracts, as the second section, the median sagittal section of the fetus intersecting the section including the head of the fetus, which is the first section, and including the reference line BL1 based on the orientation of the head of the fetus.

Hereinafter, a method for extracting the second section in the medical processing apparatus 30 will be described.

In step S15, first, the second section extraction function 353 detects the posture of the fetus included in the section including the head of the fetus. Specifically, the second section extraction function 353 detects the orientation of the head of the fetus as the posture of the fetus by obtaining a yaw angle of the head of the fetus. The yaw angle indicates an inclination (rotation) angle about an axis orthogonal to the section, that is, an axis parallel to a Z-axis illustrated in FIG. 3.

Here, the posture of the fetus is an orientation of the whole body of the fetus including the orientation of the head of the fetus and an orientation of the torso of the fetus. However, the second section extraction function 353 detects the orientation of the head of the fetus as the orientation of the whole body of the fetus by obtaining the yaw angle of the head of the fetus. This is because, since it can be assumed that the yaw angle of the head of the fetus coincides with a yaw angle of the torso in a stationary fetus in early weeks of pregnancy (for example, up to a thirteenth week of pregnancy), the orientation of the head of the fetus can be assumed as the orientation of the whole body of the fetus by obtaining the yaw angle of the head of the fetus. Then, since the fetus that is the subject is the fetus in early weeks of pregnancy, the second section extraction function 353 can detect the orientation of the head of the fetus as the orientation of the whole body of the fetus by obtaining the yaw angle of the head of the fetus.

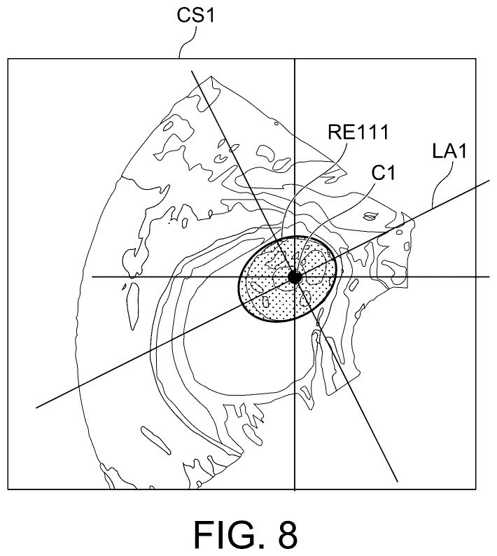

FIG. 8 is a diagram for explaining processing of detecting the posture of the fetus. As illustrated in FIG. 8, in order to identify the orientation of the whole body of the fetus from the head of the fetus, which is the predetermined site, the second section extraction function 353 detects the orientation of the head of the fetus by obtaining a major axis LA1 of an ellipse passing through a center C1 of the ellipse in the elliptically fitted region RE111 representing the fetus in the section CS1 including the head of the fetus and obtaining, as the yaw angle of the head of the fetus, an inclination of the major axis LA1 of the ellipse with respect to a horizontal direction of the section CS1.

Subsequently, in step S15, the second section extraction function 353 extracts the second section based on the posture of the fetus included in the section CS1 including the head of the fetus. Specifically, the second section extraction function 353 extracts the second section based on the orientation of the whole body of the fetus identified from the predetermined site. More specifically, the second section extraction function 353 extracts the median sagittal section based on the orientation of the head of the fetus assumed to be the orientation of the whole body of the fetus.

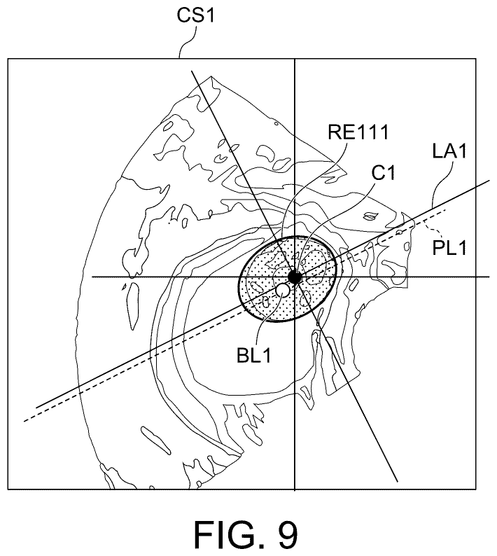

FIG. 9 is a diagram for explaining processing of extracting the second section. As illustrated in FIG. 9, the second section extraction function 353 extracts, as the second section, the median sagittal section of the fetus including the reference line BL1 by rotating by the same angle as the yaw angle of the head of the fetus, obtaining a plane PL1 intersecting the section CS1 including the head of the fetus (that is, parallel to the major axis LA1 of the ellipse) and including the reference line BL1, and cutting the three-dimensional volume data VD1 along the plane PL1.

Subsequently, as illustrated in FIG. 2, the information output function 354 in the processing circuitry 35 of the medical processing apparatus 30 outputs information based on the median sagittal section (step S17). Specifically, the information output function 354 outputs, as the information based on the median sagittal section, an image based on the median sagittal section extracted from the three-dimensional volume data VD1.

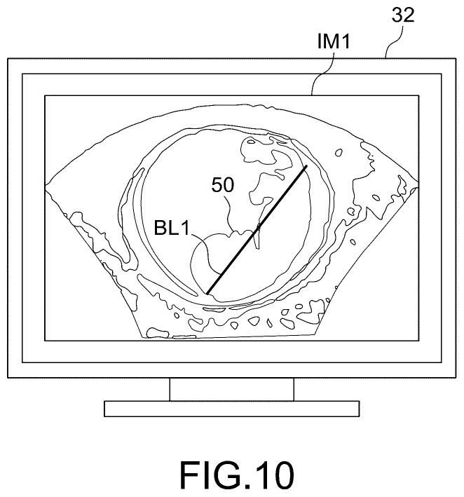

FIG. 10 is a diagram illustrating an example of information based on the second section. As illustrated in FIG. 10, the information output function 354 outputs an image IM1 based on the median sagittal section extracted from the three-dimensional volume data VD1 via the display 32. In the example illustrated in FIG. 10, the whole body 50 of the fetus is illustrated in the image IM1 based on the median sagittal section. In addition, in the example illustrated in FIG. 10, the information output function 354 superimposes the reference line BL1 acquired based on the crown-rump length L1 of the fetus on the image IM1 based on the median sagittal section and outputs the superimposed image.

Note that, in step S17 described above, although it has been described that the information output function 354 outputs the information based on the median sagittal section to the display 32, an output destination of the information based on the median sagittal section is not limited to the display 32. That is, the output destination of the information based on the median sagittal section is any destination, and the information output function 354 may output the information based on the median sagittal section to the medical image diagnostic apparatus 10 via the communication interface 34, or may output the information based on the median sagittal section to the medical image storage apparatus 20, for example.

In step S17, the information based on the median sagittal section is output, and thus, the information output processing ends.

As described above, in the medical processing apparatus 30, the crown-rump length L1 of the fetus included in the three-dimensional volume data VD1 of the fetus is detected, the reference line BL1 is acquired based on the detected crown-rump length L1 of the fetus, the section CS1 including the head of the fetus is extracted as the first section among the plurality of sections intersecting the plurality of different positions of the reference line BL1 of the fetus, the median sagittal section intersecting the section CS1 including the head of the fetus is extracted from the three-dimensional volume data VD1 based on the orientation of the fetus included in the extracted section CS1 including the head of the fetus, and the image based on the median sagittal section is output to the display 32. Thus, the user can easily generate the median sagittal section of the fetus, which is one of standard sections.

Note that, in step S13 of the information output processing described above, it has been described that the first section extraction function 352 elliptically fits the regions RE11 and RE12 representing the fetus in the plurality of sections CS1 and CS2 extracted from the three-dimensional volume data VD1 by using the elliptical template and extracts, as the first section, the section CS1, which is the section having the largest region representing the fetus among the elliptically fitted regions RE111 and RE121 in the section CS1 and the section CS2. In step S13 of the information output processing described above, the first section extraction function 352 extracts the regions RE11 and RE12 representing the fetus in the plurality of sections CS1 and CS2 extracted from the three-dimensional volume data VD1 by executing segmentation processing, and, the section CS1 having the largest region representing the fetus among the regions RE111 and RE121 representing the fetus in the extracted section CS1 and section CS2 may be extracted as the first section.

In this case, in step S15 of the information output processing described above, the second section extraction function 353 may detect the orientation of the head of the fetus by elliptically fitting the region RE111 representing the fetus in the section CS1 including the head of the fetus to a shape of the head by using the elliptical template, obtaining the major axis LA1 of the ellipse passing through the center C1 of the ellipse in the elliptically fitted region RE111 representing the fetus in the section CS1 including the head of the fetus, and obtaining, as the yaw angle of the head of the fetus, the inclination of the major axis LA1 of the ellipse with respect to the horizontal direction of the section CS1.

In addition, although it has been described that the first section extraction function 352 extracts the regions RE11 and RE12 representing the fetus in the plurality of sections CS1 and CS2 extracted from the three-dimensional volume data VD1 by executing the segmentation processing, the method for extracting the regions RE11 and RE12 representing the fetus in the plurality of sections CS1 and CS2 is not limited thereto. That is, the method for extracting the regions RE11 and RE12 representing the fetus is any method. For example, the first section extraction function 352 may extract the regions RE11 and RE12 representing the fetus by using an algorithm of extracting the regions RE11 and RE12 representing the fetus, or may extract the regions RE11 and RE12 representing the fetus by using a trained model of inputting the section extracted from the three-dimensional volume data VD1 and extracting the regions RE11 and RE12 representing the fetus included in the section.

In addition, the predetermined site of the fetus is the head of the fetus, but the predetermined site of the fetus is not limited to the head of the fetus. That is, the predetermined site of the fetus is any site, and the predetermined site of the fetus may be a site other than the head of the fetus.

Further, although it has been described that the fetus that is the subject is the fetus in early weeks of pregnancy, a target of the fetus is not limited to the fetus in early weeks of pregnancy. That is, the target of the fetus is any fetus, and the target may be a fetus in a several weeks after early weeks of pregnancy. In this case, in step S15 of the information output processing, the second section extraction function 353 may detect the posture of the fetus, for example, by obtaining the yaw angles of the head and body of the fetus.

### Modification 1

In the medical processing apparatus 30 described above, although it has been described that the first section extraction function 352 detects the crown-rump length L1 of the fetus as the reference line of the subject included in the three-dimensional volume data VD1 and extracts the first section intersecting the reference line BL1 acquired based on the detected crown-rump length L1 of the fetus, it is also possible to acquire, as the reference line of the subject included in the three-dimensional volume data VD1, a line connecting feature points of a plurality of anatomical structures of the fetus included in the three-dimensional volume data VD1, and extract the first section intersecting the line connecting the feature points of the plurality of anatomical structures of the fetus. Note that, configurations of the medical processing system 1 and the medical processing apparatus 30 are the same as those in FIG. 1, and thus, the description thereof will be omitted.

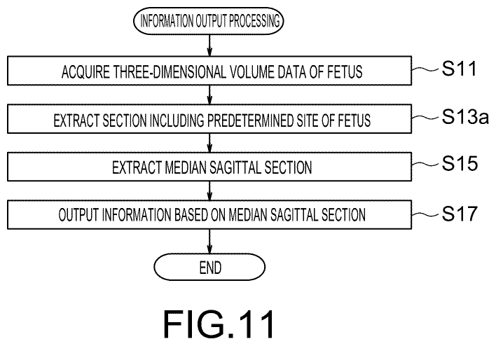

FIG. 11 is a flowchart for explaining information output processing according to Modification 1, and is a diagram corresponding to FIG. 2. In the information output processing according to Modification 1, the three-dimensional volume data of the fetus that is the subject is acquired from the medical image diagnostic apparatus 10 or the medical image storage apparatus 20, the section including the predetermined site of the fetus is extracted as the first section, the median sagittal section is extracted as the second section, or the information based on the median sagittal section is output. For example, the information output processing is processing executed in a case where the three-dimensional volume data is acquired from the medical image diagnostic apparatus 10 or the medical image storage apparatus 20. Note that, the processing in step S11 illustrated in FIG. 11 is the same as that in FIG. 2, and thus, the description thereof is omitted.

Subsequently, as illustrated in FIG. 11, the first section extraction function 352 in the processing circuitry 35 of the medical processing apparatus 30 extracts the section including the predetermined site of the fetus (step S13a). Specifically, the first section extraction function 352 according to the present modification detects the plurality of anatomical structures of the fetus that is the subject included in the three-dimensional volume data VD1, acquires the line connecting the feature points of the plurality of anatomical structures as the reference line of the subject included in the three-dimensional volume data VD1, and extracts, as the first section, the section including the predetermined site of the fetus among the plurality of sections intersecting the plurality of different positions of the line connecting the plurality of anatomical structures acquired as the reference line.

Hereinafter, a method for extracting the first section in the medical processing apparatus 30 according to the present modification will be described.

In step S13a, first, the first section extraction function 352 according to Modification 1 detects the plurality of anatomical structures of the fetus included in the three-dimensional volume data VD1 acquired in step S11. Specifically, the first section extraction function 352 according to the present modification detects the plurality of anatomical structures of the fetus included in the three-dimensional volume data VD1 by executing segmentation processing.

Note that, although it has been described that the first section extraction function 352 according to the present modification detects the plurality of anatomical structures of the fetus included in the three-dimensional volume data VD1 by executing the segmentation processing, the method for detecting the plurality of anatomical structures of the fetus included in the three-dimensional volume data VD1 is not limited thereto. That is, the method for detecting the plurality of anatomical structures of the fetus included in the three-dimensional volume data VD1 is any method. For example, the first section extraction function 352 may detect the plurality of anatomical structures by using an algorithm for detecting the anatomical structure, or may detect the plurality of anatomical structures by using a trained model for inputting the three-dimensional volume data VD1 and detecting the plurality of anatomical structures included in the three-dimensional volume data VD1.

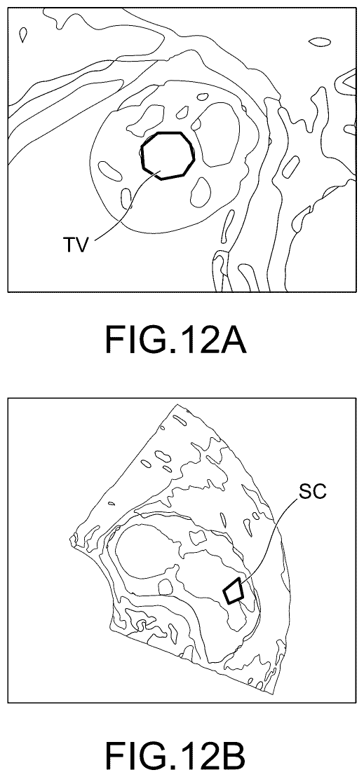

FIGS. 12A and 12B is a diagram illustrating the anatomical structure. As illustrated in FIG. 12A, the first section extraction function 352 according to the present modification detects, as one of the plurality of anatomical structures, a third ventricle TV of the fetus included in the three-dimensional volume data VD1. In addition, as illustrated in FIG. 12B, the first section extraction function 352 according to the present modification detects a sacrococcygeal part SC as one of the plurality of anatomical structures.

Subsequently, in step S13a, the first section extraction function 352 according to Modification 1 specifies the feature points of the plurality of anatomical structures. Specifically, the first section extraction function 352 according to Modification 1 identifies centers of the plurality of anatomical structures as the feature points of the plurality of anatomical structures.

Note that, although it has been described that the first section extraction function 352 according to Modification 1 specifies the centers of the plurality of anatomical structures as the feature points of the plurality of anatomical structures, the positions of the plurality of anatomical structures specified by the first section extraction function 352 according to Modification 1 are not limited to the centers. That is, the positions in the plurality of anatomical structures specified by the first section extraction function 352 according to Modification 1 are any positions, and the first section extraction function 352 according to Modification 1 may specify positions other than the centers in the plurality of anatomical structures.

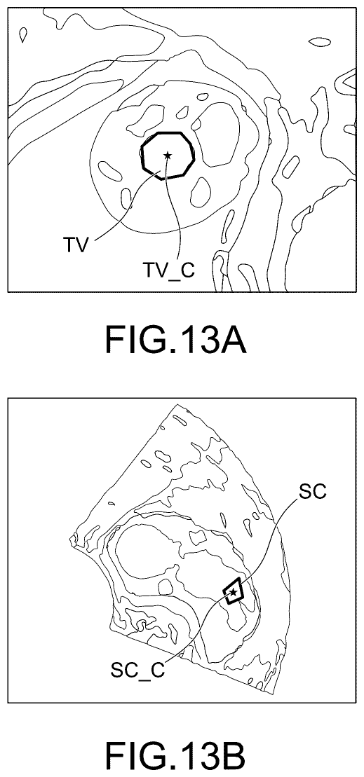

FIGS. 13A and 13B is a diagram illustrating the feature points of the anatomical structure. As illustrated in FIG. 13A, the first section extraction function 352 according to Modification 1 specifies, as the feature point, a center TV_C of the third ventricle TV, which is one of the plurality of detected anatomical structures. In addition, as illustrated in FIG. 13B, the first section extraction function 352 according to Modification 1 specifies, as the feature point, a center SC_C of the sacrococcygeal part SC, which is one of the plurality of detected anatomical structures.

Subsequently, in step S13a, the first section extraction function 352 according to Modification 1 acquires, as the reference line, the line connecting feature points of the plurality of anatomical structures. Specifically, in the example illustrated in FIGS. 13A and 13B, the first section extraction function 352 according to Modification 1 acquires, as the reference line, the line connecting the center TV_C of the third ventricle

TV and the center SC_C of the sacrococcygeal part SC.

Since the processing of step S13a after the line connecting the feature points of the plurality of anatomical structures is acquired as the reference line is equivalent to the processing of step S13, the description thereof is omitted. In addition, the processing of steps S15 and S17 after step S13a is the same as that of the first embodiment described above, and thus, the description thereof will be omitted. In step S17, the information based on the median sagittal section is output, and thus, the information output processing according to Modification 1 ends.

As described above, in the medical processing apparatus 30 according to Modification 1, the line connecting the feature points of the plurality of anatomical structures included in the three-dimensional volume data VD1 is acquired as the reference line, the section including the head of the fetus is extracted as the first section among the plurality of sections intersecting the plurality of different positions of the line connecting the feature points of the plurality of acquired anatomical structures, the median sagittal section intersecting the first section is extracted from the three-dimensional volume data VD1 based on the orientation of the fetus included in the extracted first section, and the image based on the median sagittal section is output to the display. Thus, the user can easily generate the median sagittal section of the fetus, which is one of the standard sections.

### Second Embodiment

In the medical processing apparatus 30 according to the first embodiment described above, although it has been described that, in a case where the subject is the fetus and the measurement target is the crown-rump length, the image based on the median sagittal section of the fetus is output as the information based on the second section, the information based on the subject, the measurement target, and the second section is not limited thereto. In a second embodiment, a medical processing apparatus 30 that outputs an image based on a head horizontal section as information based on a second section in a case where a subject is the head and a measurement target is the corpus callosum will be described. Note that, configurations of the medical processing system 1 and the medical processing apparatus 30 according to the second embodiment are the same as those in FIG. 1 in the first embodiment described above, and thus, the description thereof will be omitted.

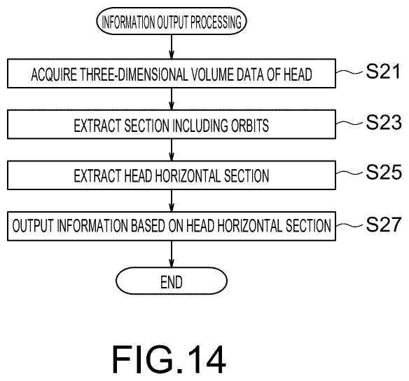

FIG. 14 is a flowchart for explaining information output processing executed in the medical processing apparatus 30 according to the second embodiment, and is a diagram corresponding to FIG. 2. In this information output processing, three-dimensional volume data of the head that is the subject is acquired from the medical image diagnostic apparatus 10 or the medical image storage apparatus 20, a section including orbits is extracted as the first section, a head horizontal section is extracted, and information based on the head horizontal section is output. For example, the information output processing is processing executed in a case where the three-dimensional volume data of the head is acquired from the medical image diagnostic apparatus 10 or the medical image storage apparatus 20.

As illustrated in FIG. 14, first, the acquisition function 351 in the processing circuitry 35 of the medical processing apparatus 30 acquires the three-dimensional volume data of the head (step S21). Specifically, the acquisition function 351 acquires the three-dimensional volume data of the head that is the subject from the medical image diagnostic apparatus 10 or the medical image storage apparatus 20 via the communication interface 34. The acquisition function 351 may directly acquire the three-dimensional volume data of the head via the communication interface 34, or the acquisition function 351 in the processing circuitry 35 may acquire the three-dimensional volume data acquired via the communication interface 34 from the storage circuitry 31 after temporarily storing the three-dimensional volume data in the storage circuitry 31.

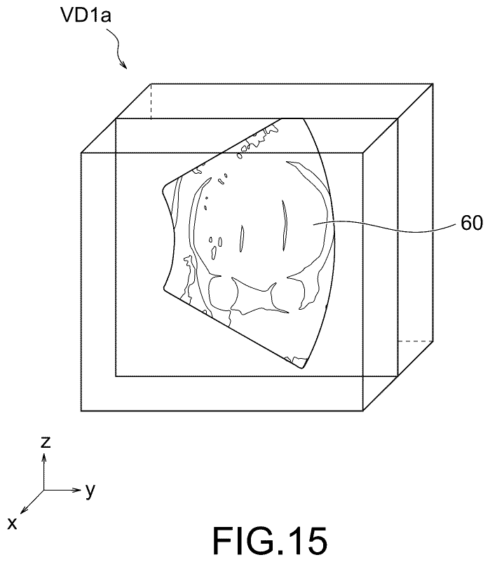

FIG. 15 is a diagram illustrating an example of the three-dimensional volume data according to the second embodiment. As illustrated in FIG. 15, the acquisition function 351 acquires three-dimensional volume data VD1a of a head 60.

Subsequently, as illustrated in FIG. 14, the first section extraction function 352 in the processing circuitry 35 of the medical processing apparatus 30 extracts the section including the orbits (step S23). Specifically, the first section extraction function 352 extracts, as the first section, the section intersecting the reference line and including the orbits.

More specifically, the first section extraction function 352 acquires the reference line based on the measurement target of the head 60 that is the subject included in the three-dimensional volume data VD1a, and extracts, as the first section, the section intersecting the reference line and including the orbits.

Hereinafter, a method for extracting the first section in the medical processing apparatus 30 will be described.

In step S23, first, the first section extraction function 352 detects the measurement target of the head included in the three-dimensional volume data VD1a acquired in step S21. Specifically, the first section extraction function 352 detects, as the measurement target, the corpus callosum of the head 60 included in the three-dimensional volume data VD1a. More specifically, the first section extraction function 352 detects the corpus callosum of the head 60 by inputting the three-dimensional volume data VD1a to the trained model and outputting the corpus callosum of the head 60 from the trained model. That is, the trained model is a model that has learned to output the corpus callosum of the head 60 in the three-dimensional volume data VD1a in response to the input of the three-dimensional volume data VD1a.

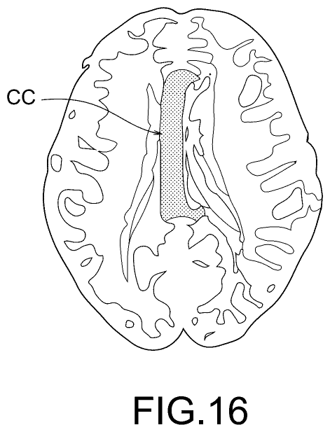

FIG. 16 is a diagram illustrating an example of the measurement target of the subject according to the second embodiment. As illustrated in FIG. 16, the first section extraction function 352 detects a corpus callosum CC of the head 60 from the three-dimensional volume data VD1a by the trained model.

Subsequently, in step S23, the first section extraction function 352 acquires the reference line based on the measurement target of the head included in the three-dimensional volume data VD1a. Specifically, the first section extraction function 352 acquires the reference line based on the corpus callosum CC that is the measurement target detected from the three-dimensional volume data VD1a. More specifically, the first section extraction function 352 acquires the reference line by linearly approximating the corpus callosum CC based on the corpus callosum CC detected from the three-dimensional volume data VD1a. The first section extraction function 352 obtains an orientation of the corpus callosum CC, that is, a tilt angle (pitch angle) of the head 60 and a yaw angle of the head 60 by acquiring the reference line. Here, the tilt angle (pitch angle) indicates an inclination angle about an axis parallel to a Y-axis illustrated in FIG. 15. In addition, the yaw angle indicates an inclination angle about an axis parallel to the Z-axis illustrated in FIG. 15.

Subsequently, in step S23, the first section extraction function 352 extracts the first section intersecting the reference line. Specifically, the first section extraction function 352 extracts, as the first section, a section intersecting the reference line acquired based on the corpus callosum CC and including the orbits. More specifically, the first section extraction function 352 extracts, as the first section, the section including the orbits, among a plurality of sections intersecting with a plurality of different positions of the reference line acquired based on the corpus callosum CC. In addition, in a case where the plurality of sections intersecting the plurality of different positions of the reference line are extracted from the three-dimensional volume data VD1a, the first section extraction function 352 extracts the plurality of sections intersecting the plurality of different positions of the reference line such that directions intersecting the reference line are equal, that is, the sections are parallel to each other.

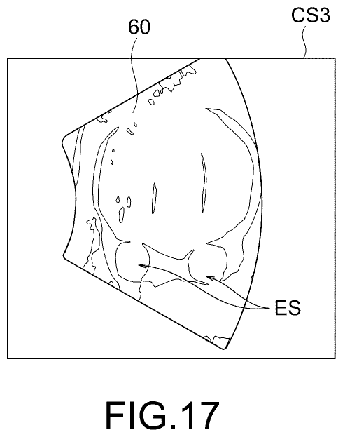

FIG. 17 is a diagram illustrating an example of the section including the orbits. As illustrated in FIG. 17, the first section extraction function 352 extracts, as the first section, a section CS3 including orbits ES from among the plurality of sections orthogonal to the plurality of different positions of the reference line based on the corpus callosum CC.

Subsequently, as illustrated in FIG. 14, the second section extraction function 353 in the processing circuitry 35 of the medical processing apparatus 30 extracts the head horizontal section (step S25). Specifically, the second section extraction function 353 extracts the head horizontal section intersecting the section CS3 including the orbits ES from the three-dimensional volume data VD1a based on a posture of the head included in the section CS3 including the orbits ES. More specifically, the second section extraction function 353 extracts a coronal section including a pair of left and right orbits from the three-dimensional volume data VD1a based on the section CS3 including the orbits, and extracts, as the second section, the head horizontal section intersecting the coronal section based on a direction in which the pair of left and right orbits included in the coronal section is connected.

Hereinafter, a method for extracting the second section in the medical processing apparatus 30 will be described.

In step S25, first, the second section extraction function 353 detects the posture of the head included in the section CS3 including the orbits ES. Specifically, the second section extraction function 353 detects the orientation of the head 60 as the posture of the head 60 by extracting the coronal section including the pair of left and right orbits ES from the three-dimensional volume data VD1a based on the section CS3 including the orbits ES extracted as the first section in step S23 and obtaining the direction in which the pair of left and right orbits ES included in the coronal section is connected.

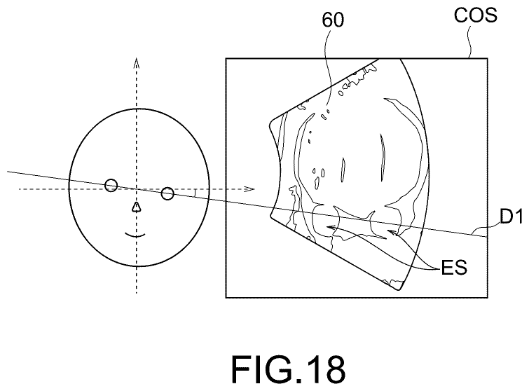

FIG. 18 is a diagram for explaining processing of detecting the posture of the head 60. As illustrated in FIG. 18, the second section extraction function 353 extracts a coronal section COS including the pair of left and right orbits ES from the three-dimensional volume data VD1a based on the section including the orbits ES extracted as the first section. In order to identify the orientation of the head 60 from the pair of left and right orbits ES which is the predetermined site, the second section extraction function 353 detects the direction of the head 60 by obtaining a direction D1 connecting the pair of left and right orbits ES included in the coronal section COS and obtaining, as a roll angle of the head 60, an inclination in the direction D1 connecting the pair of left and right orbits ES included in the coronal section COS with respect to the horizontal direction of the coronal section COS. The roll angle of the head indicates an inclination angle about an axis parallel to an X-axis illustrated in FIG. 15.

Subsequently, in step S25, the second section extraction function 353 extracts the second section based on the posture of the head 60 included in the section CS3 including the orbits ES. Specifically, the second section extraction function 353 extracts the second section based on the posture of the head identified from the predetermined site. More specifically, the second section extraction function 353 extracts the head horizontal section as the second section based on the orientation of the head 60 which is the posture of the head 60 identified from the pair of left and right orbits ES which is the predetermined site.

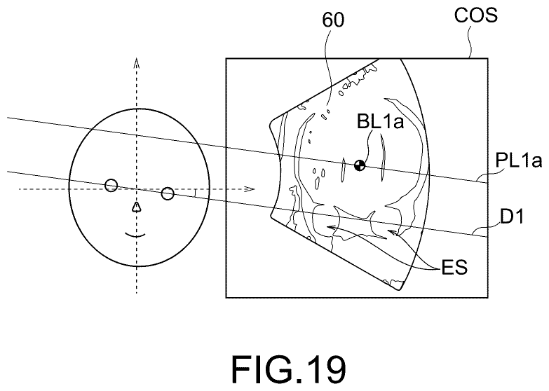

FIG. 19 is a diagram for explaining processing of extracting the second section. As illustrated in FIG. 19, the second section extraction function 353 extracts, as the second section, a head horizontal section including a reference line BL1a by rotating by the same angle as the roll angle of the head 60, obtaining a plane PL1a intersecting the coronal section COS extracted based on the section CS3 including the orbits ES (that is, parallel to the direction D1 connecting the pair of left and right orbits ES included in the coronal section COS) and including the reference line BL1a, and cutting the three-dimensional volume data VD1a with the plane PL1a.

Next, as illustrated in FIG. 14, the information output function 354 in the processing circuitry 35 of the medical processing apparatus 30 outputs the information based on the head horizontal section (step S27). Specifically, the information output function 354 outputs, as the information based on the head horizontal section, the image based on the head horizontal section extracted from the three-dimensional volume data VD1a to the display 32.

Note that, in step S27 described above, although it has been described that the information output function 354 outputs the information based on the head horizontal section to the display 32, the output destination of the information based on the head horizontal section is not limited to the display 32. That is, the output destination of the information based on the head horizontal section is any destination, and the information output function 354 may output the information based on the head horizontal section to the medical image diagnostic apparatus 10 via the communication interface 34, or may output the information based on the head horizontal section to the medical image storage apparatus 20, for example.

In step S27, the information based on the head horizontal section is output, and thus, the information output processing ends.

As described above, in the medical processing apparatus 30, the corpus callosum CC of the head 60 included in the three-dimensional volume data VD1a of the head is detected, the section CS3 including the orbits ES is extracted as the first section from among the plurality of sections intersecting the reference line BL1a based on the detected corpus callosum CC of the head 60, the head horizontal section intersecting the section CS3 including the orbits ES and including the reference line BL1a is extracted from the three-dimensional volume data VD1a based on the posture of the head 60 included in the section CS3 including the extracted orbits ES, and the image based on the head horizontal section is output to the display 32. Thus, the user can easily generate the head horizontal section, which is one of the standard sections.

### Modification 2

In the medical processing apparatus 30 according to the first and second embodiments described above, it is also possible to change the position of the reference line based on the bilaterally symmetrical anatomical structure included in the first section and extract the first section intersecting the changed reference line. Hereinafter, a case where this modification is applied to the first embodiment will be described as Modification 2, and portions different from those of the above-described first embodiment will be described. Note that, hereinafter, although a case where this modification is applied to the first embodiment will be described, this modification can also be applied to Modification 1 and the second embodiment described above.

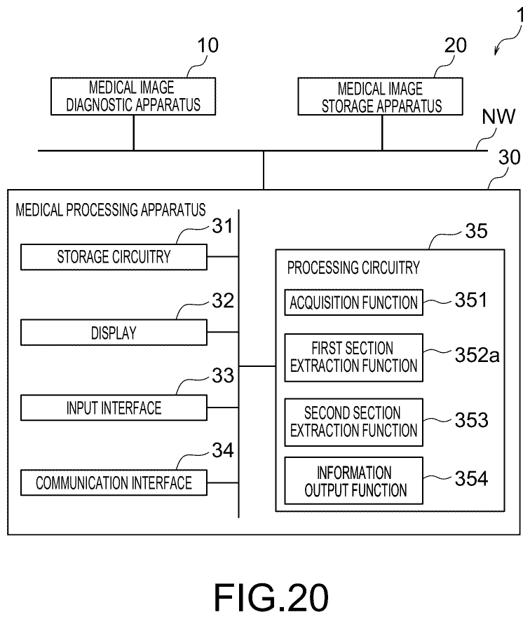

FIG. 20 is a block diagram illustrating an example of a configuration of a medical processing system 1 according to Modification 2, and is a diagram corresponding to FIG. 1. As illustrated in FIG. 20, in a medical processing apparatus 30 in the medical processing system 1 according to the present modification, since the first section extraction function of the processing circuitry 35 is different from that of the first embodiment described above, the first section extraction function is referred to as a first section extraction function 352a. Note that, configurations and functions other than the first section extraction function 352a are the same as those in FIG. 1 of the first embodiment described above, and thus, the description thereof is omitted.

The first section extraction function 352a according to the present modification changes the position of the reference line BL1 based on the bilaterally symmetrical anatomical structure included in the section CS1 including the head of the fetus, and extracts the section CS1 intersecting the changed reference line BL1 and including the head of the fetus. Here, the bilaterally symmetrical anatomical structure is a site such as a bilaterally symmetrical organ or tissue such as a lateral ventricular anterior horn, a kidney, a brain, a hand, a foot, an ear, an eye, and a lung. In the following description, a case where the bilaterally symmetrical anatomical structure is the lateral ventricular anterior horn will be described as an example.

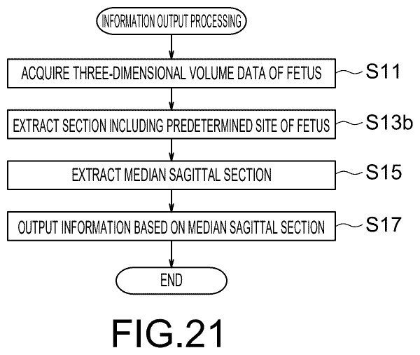

FIG. 21 is a flowchart for explaining information output processing executed in the medical processing apparatus 30 according to Modification 2, and is a diagram corresponding to FIG. 2. In the information output processing according to Modification 2, the three-dimensional volume data of the fetus that is the subject is acquired from the medical image diagnostic apparatus 10 or the medical image storage apparatus 20, the section including the predetermined site of the fetus is extracted as the first section, the median sagittal section is extracted as the second section, or the information based on the median sagittal section is output. For example, the information output processing is processing executed in a case where the three-dimensional volume data is acquired from the medical image diagnostic apparatus 10 or the medical image storage apparatus 20. Note that, the processing in step S11 illustrated in FIG. 21 is the same as that in FIG. 2, and thus, the description thereof is omitted.

Subsequently, as illustrated in FIG. 21, the first section extraction function 352a in the processing circuitry 35 of the medical processing apparatus 30 extracts the first section (step S13b). Specifically, the first section extraction function 352a extracts the first section intersecting the reference line and including the predetermined site of the fetus. More specifically, the first section extraction function 352a acquires the reference line based on the crown-rump length that is the measurement target of the fetus that is the subject included in the three-dimensional volume data VD1, and extracts, as the first section, the section intersecting the reference line and including the predetermined site of the fetus. The first section extraction function 352a detects the bilaterally symmetrical anatomical structure included in the section including the predetermined site of the fetus, determines whether or not to change the position of the reference line BL1, changes the position of the reference line based on the bilaterally symmetrical anatomical structure included in the first section, and extracts the first section intersecting the changed reference line in a case where the position of the reference line BL1 is changed.

Hereinafter, a method for extracting the first section in the medical processing apparatus 30 according to the present modification will be described. Note that, processing until the first section extraction function 352a extracts, as the first section, the section including the predetermined site of the fetus among the plurality of sections intersecting the plurality of different positions of the reference line acquired based on the crown-rump length is similar to that of the above-described first embodiment, and thus, the description thereof is omitted.

Subsequently, in step S13b, the first section extraction function 352a detects the bilaterally symmetrical anatomical structure included in the extracted section including the head of the fetus, and determines whether or not to change the position of the reference line BL1. Specifically, the first section extraction function 352a according to the present modification determines whether or not to change the position of the reference line by detecting the bilaterally symmetric anatomical structure included in the section including the head of the fetus by using segmentation processing and determining whether or not the detected bilaterally symmetric anatomical structure included in the section including the head of the fetus is bilaterally symmetric in the section including the head of the fetus.

Note that, although it has been described that the first section extraction function 352 according to the present modification detects the plurality of anatomical structures of the fetus included in the three-dimensional volume data VD1 by executing the segmentation processing, the method for detecting the plurality of anatomical structures of the fetus included in the three-dimensional volume data VD1 is not limited thereto. That is, the method for detecting the plurality of anatomical structures of the fetus included in the three-dimensional volume data VD1 is any method. For example, the first section extraction function 352 may detect the plurality of anatomical structures by using an algorithm for detecting the anatomical structure, or may detect the plurality of anatomical structures by using a trained model for inputting the three-dimensional volume data VD1 and detecting the plurality of anatomical structures included in the three-dimensional volume data VD1.

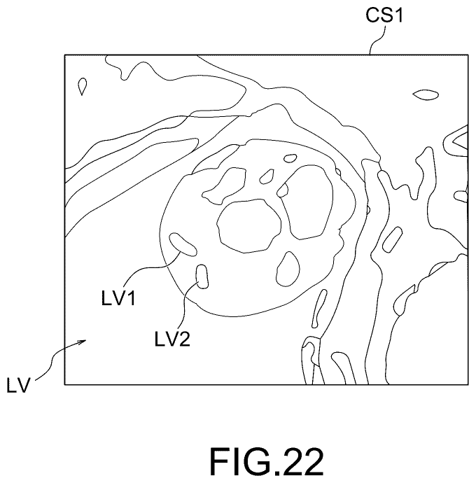

FIG. 22 is a diagram illustrating an example of the bilaterally symmetrical anatomical structure according to Modification 2. As illustrated in FIG. 22, the first section extraction function 352a according to the present modification detects, as the bilaterally symmetrical anatomical structure, lateral ventricular anterior horns LV included in the section CS1 including the head of the fetus. The first section extraction function 352a determines whether or not the position of the reference line BL1 is changed by determining whether or not the detected left and right lateral ventricular anterior horns LV included in the section CS1 including the head of the fetus are bilaterally symmetrical in the first section.

Note that, although it has been described that the first section extraction function 352a according to the present modification detects the lateral ventricular anterior horns LV as the bilaterally symmetrical anatomical structure from the section CS1 including the head of the fetus, the bilaterally symmetrical anatomical structure detected from the section CS1 including the head of the fetus by the first section extraction function 352a is not limited to the lateral ventricular anterior horns. That is, any bilaterally symmetrical anatomical structure is detected from the section CS1 including the head of the fetus by the first section extraction function 352a.

In a case where the left and right lateral ventricular anterior horns LV are bilaterally symmetrical in the section including the head of the fetus, the first section extraction function 352a according to the present modification does not change the position of the reference line BL1. On the other hand, in a case where the left and right lateral ventricular anterior horns LV are not bilaterally symmetrical in the section including the head of the fetus, the first section extraction function 352a according to the present modification changes the position of the reference line BL1 based on the left and right lateral ventricular anterior horns LV included in the section including the head of the fetus, and extracts the section including the head of the fetus intersecting the changed reference line.

The processing in steps S15 and S17 after step S13b is the same as that in the first embodiment described above, and thus, the description thereof will be omitted. Then, in step S17, the information based on the second section is output, and thus, the information output processing ends.

As described above, in the medical processing apparatus 30 according to the present modification, the position of the reference line BL1 is changed based on the bilaterally symmetrical anatomical structure included in the first section, and the first section intersecting the changed reference line is extracted. Thus, a more accurate median sagittal section of the fetus can be easily generated.

### Modification 3

In the medical processing system 1 according to the first and second embodiments, Modification 1, and Modification 2 described above, the configuration of the medical processing apparatus 30 can also be applied to the medical image diagnostic apparatus 10 or the medical image storage apparatus 20. In that case, for example, functions equivalent to the acquisition function 351, the first section extraction function 352 or 352a, the second section extraction function 353, and the information output function 354 described above are implemented in a processing circuitry included in the medical image diagnostic apparatus 10 or a processing circuitry included in the medical image storage apparatus 20.

### Other Modifications

In the medical processing apparatus 30 of the medical processing system 1 according to the first and second embodiments and Modifications 1 to 3, although it has been described that, in step S13, S13a, or S13b of the information output processing, the first section extraction function 352 or 352a extracts the first section orthogonal to the reference line BL1 or BL1a, the first section may not be orthogonal to the reference line BL1 or BL1a. That is, any angle is formed by the reference line BL1 or BL1a and the first section, and the first section extraction function 352 or 352a may extract the first section intersecting the reference line BL1 or BL1a.

In addition, in the medical processing apparatus 30 of the medical processing system 1 according to the first and second embodiments and Modifications 1 to 3 described above, although it has been described that, in step S13, S13a, or S13b of the information output processing, the first section extraction function 352 or 352a detects the measurement target of the subject by using the trained model and logic, the method for detecting the measurement target of the subject is not limited thereto. That is, the method for detecting the measurement target of the subject is any method. For example, the first section extraction function 352 may detect the measurement target of the subject from the three-dimensional volume data VD1 or VD1a by using only the logic of detecting the measurement target of the subject, or may receive the input operation related to the detection of the measurement target of the subject from the user and may detect the measurement target of the subject from the three-dimensional volume data VD1 or VD1a according to the received input operation.

In addition, although it has been described that the subject is the fetus in the first embodiment described above and the subject is the head in the second embodiment described above has been described as an example, the subject is not limited thereto. That is, the subject is any subject, and the subject may be, for example, a heart or the like.

In addition, in the second embodiment described above, although it has been described that the second section extraction function 353 extracts, as the second section, the head horizontal section including the reference line BL1a by rotating by the same angle as the roll angle of the head 60, obtaining the plane PL1a intersecting the coronal section COS extracted based on the section CS3 including the orbits ES and including the reference line BL1a, and cutting the three-dimensional volume data VD1a with the plane PL1a, the head horizontal section is not limited to the case of including the reference line BL1a. That is, the second section extraction function 353 may extract, as the second section, the head horizontal section not including the reference line BL1a by rotating by the same angle as the roll angle of the head 60, obtaining the plane intersecting the coronal section COS extracted based on the section CS3 including the orbits ES and not including the reference line BL1a, and cutting the three-dimensional volume data VD1a on the plane.

Note that the word “processor” used in above descriptions means circuits such as, for example, a Central Processing Unit (CPU), a Graphics Processing Unit (GPU), an Application Specific Integrated Circuit (ASIC), a programmable logic device (for example, a Simple Programmable Logic Apparatus (SPLD), a Complex Programmable Logic Apparatus (CPLD), and a Field Programmable Gate Array (FPGA)). The processor executes functions by reading and executing programs stored in the storage circuitry 31. Note that programs may be configured to be directly integrated in the processor instead of being storing in the storage circuitry 31. In this case, the processor realizes functions by reading and executing programs stored in the circuit. Note that the processor is not limited to the case arranged as a single processor circuit, but may be configured as a single processor by combining a plurality of independent circuits to realize functions. Furthermore, a plurality of component elements in FIG. 1 may be integrated into one processor to realize the functions.

While certain embodiments have been described, these embodiments have been presented by way of example only and are not intended to limit the scope of the inventions. The embodiments may be in a variety of other forms. Furthermore, various omissions, substitutions and changes may be made without departing from the spirit of the inventions. The embodiments and their modifications are included in the scope and the subject matter of the invention, and at the same time included in the scope of the claimed inventions and their equivalents.

1. A medical processing apparatus comprising:
a processing circuitry configured to:
acquire three-dimensional volume data of a subject;
extract a first section intersecting a reference line of the subject included in the three-dimensional volume data;
extract a second section intersecting the first section from the three-dimensional volume data based on a posture of the subject included in the first section; and
output information based on the second section.

2. The medical processing apparatus of claim 1, wherein the processing circuitry is further configured to extract a first section intersecting the reference line and including a predetermined site of the subject, and
the second section extraction unit extracts the second section based on the posture of the subject identified from the predetermined site.

3. The medical processing apparatus of claim 2, wherein the processing circuitry is further configured to extract, as the first section, a section including the predetermined site of the subject among a plurality of sections intersecting a plurality of different positions of the reference line.

4. The medical processing apparatus of claim 1, wherein:
the subject is a fetus, and
the processing circuitry is further configured to acquire the reference line based on a measurement target of the fetus included in the three-dimensional volume data, and extract the first section intersecting the reference line.

5. The medical processing apparatus of claim 4, wherein:
the measurement target is a crown-rump length (CRL), and
the processing circuitry is further configured to acquire the reference line based on the crown-rump length detected from the three-dimensional volume data.

6. The medical processing apparatus of claim 5, wherein the processing circuitry is further configured to:
extract, as the first section, a section intersecting the reference line and including a head of the fetus; and
extract the second section based on a posture of the fetus identified from the head of the fetus.

7. The medical processing apparatus of claim 6, wherein the processing circuitry is further configured to extract, as the first section including the head of the fetus, a section having a largest region representing the fetus, among a plurality of sections intersecting a plurality of different positions of the reference line.

8. The medical processing apparatus of claim 6, wherein the processing circuitry is further configured to detect an orientation of the head of the fetus as the posture of the fetus, and extract, as the second section, a median sagittal section of the fetus intersecting the first section and including the reference line based on the orientation of the head of the fetus.

9. The medical processing apparatus of claim 1, wherein:
the subject is a head, and
the processing circuitry is further configured to acquire the reference line based on a measurement target of the head included in the three-dimensional volume data.

10. The medical processing apparatus of claim 9, wherein:
the measurement target is a corpus callosum, and
the processing circuitry is further configured to acquire the reference line based on the corpus callosum detected from the three-dimensional volume data.

11. The medical processing apparatus of claim 10, wherein the processing circuitry is further configured to extract, as the first section, a section intersecting the reference line and including orbits.

12. The medical processing apparatus of claim 11, wherein the processing circuitry is further configured to extract a coronal section including a pair of left and right orbits from the three-dimensional volume data based on the section including the orbits, and extract, as the second section, a head horizontal section intersecting the coronal section based on a direction in which the pair of left and right orbits included in the coronal section is connected.

13. The medical processing apparatus of claim 1, wherein the processing circuitry is further configured to detect a plurality of anatomical structures of the subject and acquire, as the reference line, a line connecting feature points of the plurality of anatomical structures.

14. The medical processing apparatus of claim 1, wherein the processing circuitry is further configured to change a position of the reference line based on a bilaterally symmetric anatomical structure included in the first section and extract the first section intersecting the changed reference line.

15. The medical processing apparatus of claim 1, wherein the processing circuitry is further configured to detect a measurement target of the subject by inputting the three-dimensional volume data to a trained model, detecting a predetermined region of the subject by the trained model, and obtaining information regarding the measurement target of the subject from the predetermined region by a logic.

16. A medical processing method comprising:
acquiring three-dimensional volume data of a subject;
extracting a first section intersecting a reference line of a subject included in the three-dimensional volume data;
extracting a second section intersecting the first section from the three-dimensional volume data based on a posture of the subject included in the first section; and
outputting information based on the second section.
